(19) United States
(12) Patent Application Publication
(10) Pub. No.: US 2007/0100411 A1
(43) Pub. Date: May 3, 2007

(54) Implantable medical electrical stimulation lead fixation method and apparatus

(75) Inventor: Eric H. Bonde, Minnetonka, MN (US)

Correspondence Address: MEDTRONIC, INC., 710 MEDTRONIC PARK, MINNEAPOLIS, MN, 55432-9924, US

(73) Assignee: Medtronic, Inc., Minneapolis, MN (US)

(21) Appl. No.: 11/260,350

(22) Filed: Oct. 27, 2005

(51) Int. Cl. A61N 1/05

(52) U.S. Cl. 607/126

(57) ABSTRACT

An implantable medical electrical lead for electrical stimulation of body tissue that includes a lead body extending between lead proximal and distal ends, at least one tine element that includes at least one flexible, pliant, tine, that is adapted to be folded inward and temporarily secured against the lead body using a temporary fixative, and at least one electrode, wherein the at least one electrode is distal of the at least one tine element on the lead body. A medical electrical stimulation system that includes an implantable pulse generator for providing medical electrical stimulation, and a medical electrical lead coupled to the implantable pulse generator for electrical stimulation of body tissue, the medical electrical lead including a lead body extending between lead proximal and distal ends. at least one tine element that includes at least one flexible, pliant, tine, that is adapted to be folded inward and temporarily secured against the lead body using a temporary fixative, and at least one electrode, wherein the at least one electrode is distal of the at least one tine element on the lead body.

### FIELD OF THE INVENTION

This invention relates generally to a method and apparatus that allows for stimulation of body tissue. More specifically, this invention relates to an implantable medical electrical lead having at least one stimulation electrode adapted to be implanted and a fixation mechanism for providing chronic stability of the stimulation electrode and lead.

### BACKGROUND OF THE INVENTION

Pelvic floor disorders such as, urinary incontinence, urinary urge/frequency, urinary retention, pelvic pain, bowel dysfunction (constipation, diarrhea), erectile dysfunction, are bodily functions influenced by the sacral nerves. Specifically, urinary incontinence is the involuntary control over the bladder that is exhibited in various patients. Incontinence is primarily treated through pharmaceuticals and surgery. Many of the pharmaceuticals do not adequately resolve the issue and can cause unwanted side effects, and a number of the surgical procedures have a low success rate and are not reversible. Several other methods have been used to control bladder incontinence, for example, vesicostomy or an artificial sphincter implanted around the urethra. These solutions have drawbacks well known to those skilled in the art. In addition, some disease states do not have adequate medical treatments.

The organs involved in bladder, bowel, and sexual function receive much of their control via the second, third, and fourth sacral nerves, commonly referred to as S2, S3 and S4 respectively. Electrical stimulation of these various nerves has been found to offer some control over these functions. Several techniques of electrical stimulation may be used, including stimulation of nerve bundles within the sacrum. The sacrum, generally speaking, is a large, triangular bone situated at the lower part of the vertebral column, and at the upper and back part of the pelvic cavity. The spinal canal runs throughout the greater part of the sacrum. The sacrum is perforated by the anterior and posterior sacral foramina that the sacral nerves pass through.

Neurostimulation leads have been implanted on a temporary or permanent basis having at least one stimulation electrode positioned on and near the sacral nerves of the human body to provide partial control for bladder incontinence. Temporary sacral nerve stimulation is accomplished through implantation of a temporary neurostimulation lead extending through the skin and connected with a temporary external pulse generator as described for example in commonly assigned U.S. Pat. Nos. 5,957,965 ad 6,104,960. A permanent neurostimulator is implanted if stimulation is efficacious and it is possible to do so in the particular patient. Permanent implantation is accomplished by implanting a permanent neurostimulation lead, extending the proximal portion of the lead body subcutaneously, and connecting its proximal end with an implantable pulse generator (IPG) implanted subcutaneously.

A problem associated with implantation of permanent and temporary neurostimulation leads involves maintaining the discrete ring-shaped electrode(s) in casual contact, that is in a location where slight contact of the electrode with the sacral nerve may occur or in close proximity to the sacral nerve to provide adequate stimulation of the sacral nerve, while allowing for some axial movement of the lead body. Typically, physicians spend a great deal of time with the patient under a general anesthetic placing the leads due to the necessity of making an incision exposing the foramen and due to the difficulty in optimally positioning the small size stimulation electrodes relative to the sacral nerve. The patient is thereby exposed to the additional dangers associated with extended periods of time under a general anesthetic. Movement of the lead, whether over time from suture release or during implantation during suture sleeve installation, is to be avoided. As can be appreciated, unintended movement of any object positioned proximate a nerve may cause unintended nerve damage. Moreover reliable stimulation of a nerve requires consistent nerve response to the electrical stimulation that, in turn, requires consistent presence of the stimulation electrode proximate the sacral nerve. But, too close or tight a contact of the electrode with the sacral nerve can also cause inflammation or injury to the nerve diminishing efficacy and possibly causing patient discomfort.

Once the optimal electrode position is attained, it is necessary to fix the lead body to retard lead migration and dislodgement of the electrodes from the optimal position. This can be accomplished by employing sutures or a sacral lead fixation mechanism, an example of which is described in commonly assigned U.S. Pat. No. 5,484,445. Another example of a lead that includes a fixation mechanism can be found in commonly assigned U.S. patent application Ser. No. 10/004,732, the disclosure of which is incorporated herein by reference.

Although the fixation mechanisms of the above referenced application are a significant advance over the prior art, there are still further advantages to be gained. For example, it can be difficult to place those leads because once the tines are released from the dilator sheath, the tines deploy and it becomes impossible to retract the lead body and position it again. Furthermore, the lead of the above referenced application cannot be configured with a forward facing tine, which may be advantageous in order to decrease possible forward lead migration.

### SUMMARY OF THE INVENTION

The invention provides an implantable medical electrical lead for electrical stimulation of body tissue that includes a lead body extending between lead proximal and distal ends, at least one tine element that includes at least one flexible, pliant, tine, that is adapted to be folded inward and temporarily secured against the lead body using a temporary fixative, and at least one electrode, wherein the at least one electrode is distal of the at least one tine element on the lead body.

The invention provides a medical electrical stimulation system that includes an implantable pulse generator for providing medical electrical stimulation, and a medical electrical lead coupled to the implantable pulse generator for electrical stimulation of body tissue, the medical electrical lead including a lead body extending between lead proximal and distal ends. at least one tine element that includes at least one flexible, pliant, tine, that is adapted to be folded inward and temporarily secured against the lead body using a temporary fixative, and at least one electrode, wherein the at least one electrode is distal of the at least one tine element on the lead body.

The invention provides a method of providing electrical stimulation of body tissue at a stimulation site employing an implantable pulse generator that includes providing an implantable medical lead that includes a lead body extending between lead proximal and distal ends, at least one tine element that includes at least one flexible, pliant, tine, that is adapted to be folded inward and temporarily secured against the lead body using a temporary fixative, at least one electrode, wherein the at least one electrode is distal of the at least one tine element on the lead body, at least one proximal connector element formed in a connector array in a proximal segment of the lead body, percutaneously introducing the implantable medical lead adjacent to the stimulation site, allowing the temporary fixative to dissolve, thereby allowing the at least one tine element to fold outward, and coupling the at least one proximal connector element with the implantable pulse generator.

The full range of advantages and features of this invention are only appreciated by a full reading of this specification and a full understanding of the invention. Therefore, to complete this specification, a detailed description of the invention and the preferred embodiments follow, after a brief description of the drawings, wherein additional advantages and features of the invention are disclosed.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

Embodiments of the invention are illustrated in the drawings, wherein like reference numerals refer to like elements in the various views. Furthermore, it will be understood by one of skill in the art that the drawings are not drawn to scale.

FIG. 1 is a plan view of one embodiment of a stimulation lead of the present invention having a tine element array and stimulation electrode array in a distal portion of the lead body.

FIG. 2 is an expanded perspective view of the tine element array and stimulation electrode array in the distal portion of the lead body of FIG. 1.

FIG. 3 is an expanded perspective view of one embodiment of a tine element employed in the lead of FIGS. 1 and 2.

FIG. 4 is an expanded perspective view of another embodiment of a tine element employed in the lead of FIGS. 1 and 2.

FIG. 5 is an expanded perspective view of yet another embodiment of a tine element array of a lead body.

FIG. 6 is an expanded perspective view of yet another embodiment of a tine element array of a lead body.

FIG. 7 is a cross-section view of the sacrum schematically illustrating an initial step of implanting a sacral nerve stimulation lead of the present invention with tines constrained within an introducer lumen;

FIG. 8 is a cross-section view of the sacrum schematically illustrating a further step of implanting a sacral nerve stimulation lead of the present invention extending the stimulation electrodes through a foramen;

FIG. 9 is a cross-section view of the sacrum schematically illustrating a further step of implanting a sacral nerve stimulation lead of the present invention retracting the introducer to release the tines in subcutaneous tissue;

FIG. 10 is a cross-section view of the sacrum schematically illustrating a further step of implanting a sacral nerve stimulation lead of the present invention subcutaneously routing the proximal portion of the lead body to the implantation site of the neurostimulator IPG;

### DETAILED DESCRIPTION OF THE INVENTION

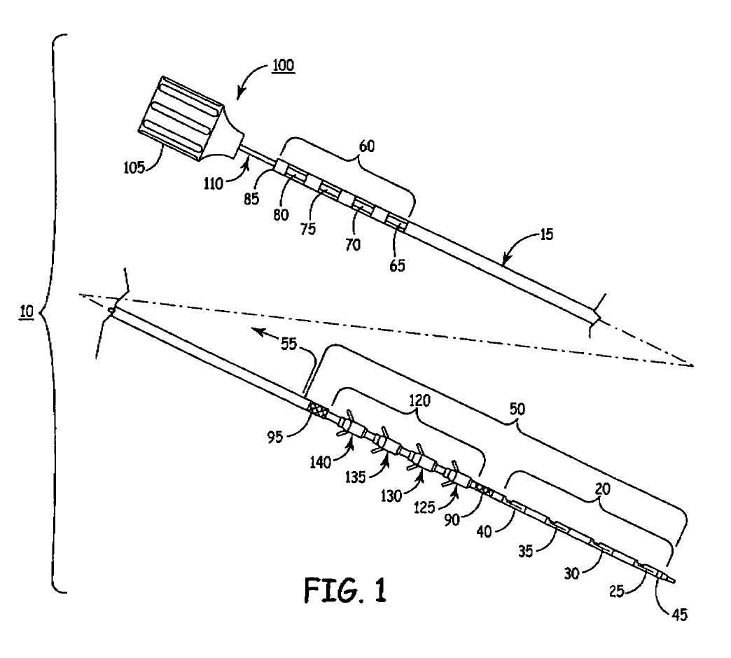

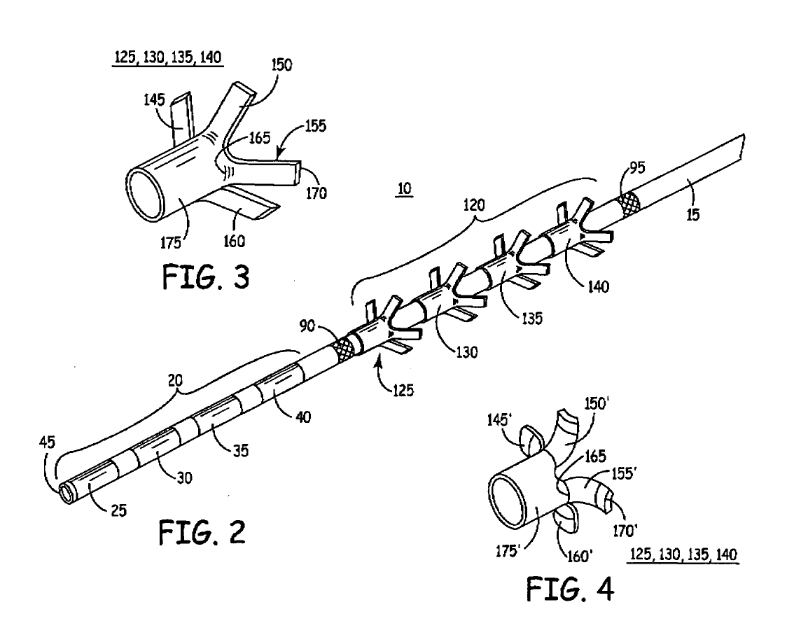

Referring to FIGS. 1 and 2, an example of an implantable medical lead 10 that allows for non-direct contact stimulation of various nerves, including for example the sacral nerve, includes a lead body 15. In one embodiment, the lead body outer diameter is in the range of about 0.5 mm to about 2 mm, and the lead 10 is about 28.0 cm long. In another embodiment, the lead body 15 has an outer diameter of about 1.3 mm. In one embodiment, the lead body 15 has, for example, four ring-shaped electrodes 25, 30, 35, and 40 in an electrode array 20 extending proximally from the lead distal end 45. The electrode array 20 extends proximally longitudinally for a length of about 25.0 mm from the distal end 45. In one embodiment, the electrodes 25, 30, 35 and 40 are made of a solid surface, bio-compatible material, e.g., a tube formed of platinum, platinum-iridium alloy, or stainless steel, of about 3.0 mm in length that does not degrade when it is separated by shorter insulator bands and electrical stimulation is delivered through it.

Each stimulation electrode 25, 30, 35, and 40 is electrically coupled to the distal end of a coiled wire lead conductor within the elongated lead body 15 that extends proximally through the distal portion 50 and through the proximal portion 55 of the lead body 15. The proximal ends of the separately insulated lead conductors are each coupled to respective ring-shaped connector elements 65, 70, 75, and 80 in a proximal connector element array 60 along the proximal portion 55 of the lead body 15 adjacent the lead proximal end 85. In one embodiment, the conductor wires are formed of an MP35N alloy and are insulated from one another within an insulating polymer sheath such as polyurethane, fluoropolymer or silicone rubber for example. The lead conductor wires are separately insulated by an insulation coating and can be wound in a quadra-filar manner having a common winding diameter within the outer sheath. The coil formed by the coiled wire conductors defines a lead body lumen of the lead body 15. It will be understood that a further inner tubular sheath could be interposed within the aligned wire coils to provide the lead body lumen.

The connector elements 65, 70, 75, and 80 can be adapted to be coupled with a neurostimulator IPG, additional intermediate wiring, or other stimulation device adapted to be implanted subcutaneously. An example of such an implantable pulse generator is the MEDTRONIC INTERSTIM® Neurostimulator Model 3023. Electrical stimulation pulses generated by the neurostimulator IPG are applied to a nerve, such as the sacral nerve, through one or more of the stimulation electrodes 25, 30, 35 and 40 in either a unipolar or bipolar stimulation mode.

The axial lead body lumen (not shown) extends the length of the lead body 15 between a lumen proximal end opening at lead proximal end 85 and a lumen distal end opening at lead distal end 45. The straight wire 110 attached to the handle 105 of a guide wire or stiffening stylet 100 can be inserted through the lead body lumen to assist in implanting the lead 10 as described further below. In one embodiment the stylet wire 110 can be made of solid wire such as tungsten or stainless steel.

A fixation mechanism is formed on the lead body 15 proximal to the electrode array 20 in the distal lead portion 50 that is adapted to be implanted in and engage subcutaneous tissue to inhibit axial movement of the lead body 15 and dislodgement of the stimulation electrodes 25, 30, 35 and 40. The fixation mechanism comprises one or more tine elements. The embodiment depicted in FIGS. 1 and 2 includes four tine elements, tine elements 125, 130, 135 and 140 arrayed in a tine element array 120 in the distal lead portion 50 of the lead body 15.

FIG. 2 depicts a more detailed view of a portion of the lead 10 depicted in FIG. 1. In this embodiment there are four stimulation electrodes 25, 30, 35, and 40; and four tine elements 125, 130, 135, and 140. Each tine element 125, 130, 135 and 140 comprises at least one flexible, pliant, tine, and four such tines 145, 150, 155 and 160 or 145′, 150′, 155′ and 160′, are depicted in these examples. Each tine, e.g., tine 155 in FIG. 3 or 155′ in FIG. 4, has a tine width and thickness and extends through a tine length from an attached tine end 165 to a free tine end 170. The attached tine end 165 is attached to the lead body 15 from a tine attachment site and supports the tine extending outwardly of the lead body 15 and proximally toward the lead proximal end 85. The tine end 165 can be attached to the lead body 15 as would be known to one of skill in the art having read this specification. Examples of ways of attaching the tine end 165 to the lead body 15 include, but are not limited to spot welding, and the use of adhesives. In one embodiment, the tine end 165 is adhered to the lead body 15 using an adhesive.

In the depicted embodiments, the tine elements 125, 130, 135 and 140 or 125′, 130′, 135′ and 140′ can include a tine mounting band 175 or 175′ that encircles the lead body with the tines extending from respective attached tine ends or roots disposed apart from one another. In one embodiment, the tines are equally spaced around the tine mounting band 175 or 175′. The four tines 145, 150, 155 and 160 or 145′, 150′, 155′ and 160′, have a tine thickness that enables folding of the tines against the lead body in the space between the tine mounting band and the adjoining proximal tine mounting band depicted in FIGS. 1 and 2.

In one embodiment, the mounting band 175, 175′ has an outside diameter of about 0.04 to about 0.10 inches and is about 0.02 to about 0.10 inches long. In another embodiment, the outside diameter is about 0.06 inches and is about 0.08 inches long. In another embodiment, the outside diameter is about 0.062 inches and the length is about 0.076 inches. In one embodiment, each tine is between about 0.005 inches and about 0.02 inches thick, has a length from about 0.02 inches to about 0.12 inches, and has a width between about 0.02 inches and about 0.07 inches. In another embodiment each tine is about 0.013 inches thick, about 0.07 inches long, and is about 0.035 inches wide. In one embodiment, each tine extends radially outward at an angle between about 30 degrees and about 90 degrees to the axis of the lead body and mounting band 175, 175′. In another embodiment, each tine extends radially outward and at about 45° to the axis of the lead body and the mounting band 175, 175′. One of skill in the art, having read this specification will understand that the particular dimensions of the tines may be dictated at least in part by the dimensions and ultimate implanted location of the lead that the tines will be adhered to.

In one embodiment, the tine elements are formed of a bio-compatible plastic, e.g., medical grade silicone rubber or polyurethane that can be formulated to exhibit a desired degree of stiffness or flexibility. In another embodiment, the tine elements are formed of a superelastic alloy material.

The tines are adapted to be folded inward against the lead body 15 and temporarily secured there using a temporary fixative. The temporary fixative functions to temporarily hold the tine elements inward against the lead body 15 so that the lead can be inserted in the vicinity of the nerve. In one embodiment the tines are held inward such that the tine free ends of more distal tines of more distal tine elements are urged toward or alongside the attached tine ends of the adjacent more proximal tines of more proximal tine elements, and the folded tines do not overlap one another. In the previously referenced patent application (App Ser. No. 10/004,732), the tines were held inward against the lead body when fitted into and constrained by the lumen of an introducer. Contrary to that, the present invention holds the tines inward using the temporary fixative.

In one embodiment, the temporary fixative releases the tines from the inward position when it dissolves, breaks down, or is broken down by the tissue surrounding it. Generally, the temporary fixative can include water soluble, biocompatible adhesives. Generally, a material that retains the tines inward against the lead body 15 for a period of at least about 1 minute to at least about 3 weeks or longer can be used as a temporary fixative. In another embodiment, a material that retains the tines inward against the lead body 15 for at least about 5 minutes up to about 12 hours can be used as a temporary fixative.

In one embodiment different materials can be used as temporary fixative in the same lead. This could allow different tines to be deployed at different times. Alternatively, the same material could be used, but different concentrations, amounts, or solvents could be used to modify the deployment time of the various tines in one lead. This may be useful in embodiments having more than one or multiple tines on one lead. In embodiments where rapid tine deployment is desired, rapidly dissolving temporary fixative material could be utilized. Alternatively, the region containing the temporary fixative (constraining the tines that are to be deployed quickly) can be flushed with saline for example through a delivery sheath. Additionally, mechanical agitation or vibration can accelerate the degradation of the fixative.

Examples of materials that can be used as the temporary fixative include, but are not limited to natural materials and synthetic materials. Examples include, but are not limited to sugars such as glucose or dextrose, a sugar alcohol such as mannitol, sugar celluloses such as polysaccharide glucose, protein solutions such as albumin, and possibly naturally derived and modified acrylic or alkyd resins or varnishes (assuming biocompatibility). An example of these may be acrylics derived from grains or alkyds derived from coconuts. Additionally, formulations that can be degraded by hydrolization may be employed. These include, but are not limited to, formulations such as polyglycolic acid, polyglactin, polydioxone, and polyglyconate. Other examples of natural materials include materials from animal intestines such as catgut or isinglass or a fixative such as beeswax. In one embodiment, the temporary fixative is a simple sugar solution or sugar alcohol that is degraded by solubility and metabolization. In another embodiment, the temporary fixative is a formulation of polyglactin.

In one embodiment, the temporary fixative is applied to the entire lead body 15 or the majority of the lead body 15. In another embodiment, the temporary fixative is applied only to the distal end 50 of the lead body 15. In yet another embodiment, the temporary fixative is applied to only a portion of the distal end 50 of the lead body 15. In a further embodiment, the temporary fixative is applied only to the region of the tine element array 120 of the distal end 50.

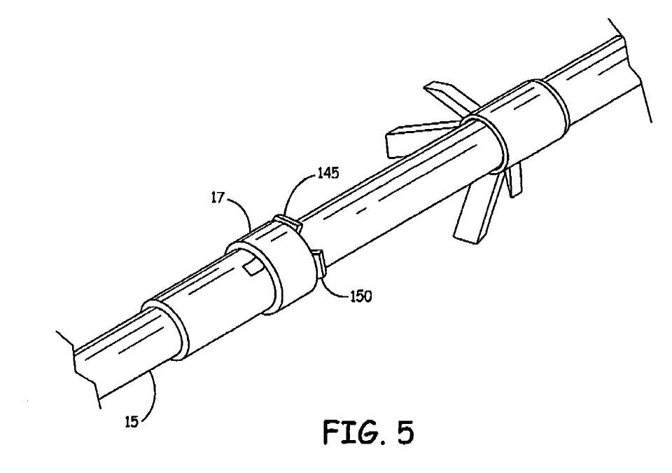

In yet another embodiment of the invention depicted in FIG. 5, only a small area (for example, the area directly covering the tines) has temporary fixative applied thereto. The embodiment depicted in FIG. 5 includes a band 17 of temporary fixative that binds the tines 145, 150 to the lead body 15. In one embodiment the band 17 secures the tines 145, 150 to the lead body 15 by its physical and material strength. When the band 17 degrades or dissolves the tines 145, 150 are released. As discussed above, the band 17 can be made of the temporary fixative materials discussed above. In one embodiment the temporary fixative material for the band 17 can include, but is not limited to materials that are similar to those materials that widely commercially available absorbable sutures are made from. Examples of these materials include, but are not limited to natural materials made from animal intestines and synthetic formulations such as polyglycolic acid, polyglactin, polydioxone, and polyglyconate for example.

When manufacturing a lead in accordance with this invention, the lead body, including the electrodes, tines, etc. can be manufactured as was known to one of skill in the art, having read this specification, at the time of the invention. After the lead was manufactured, the next step would be to fold the tines inward and apply the temporary fixative. One method of accomplishing this would be to house the lead in a lumen like structure that is similar to the lead introducer as used in commonly owned U.S. application Ser. No. 10/004,732 and apply the temporary fixative to the inside of the lead introducer. Another method would be to apply the temporary fixative to the inside of a hollow lumen or tube that has a slightly larger diameter than the tine element array 120 and a length that at least spans the distance from the fixed end of the most distal tine element (125 in FIGS. 1 and 2) to the free end of the most proximal tine element (140 in FIGS. 1 and 2).

In one embodiment, the inside of the lumen is coated or treated so that the temporary fixative will not adhere to the lumen. In another embodiment, the temporary fixative could be applied to the tine element array 120 region and then a structure, such as the hollow lumens discussed above, could be put in place to secure the tine elements inward while the temporary fixative is dried or cured. This embodiment could also include the step of coating or treating the inside of the hollow lumen so that the temporary fixative does not adhere to it.

In manufacturing the embodiment depicted in FIG. 5, the band 17 may be expanded over a tapered mandrel and then released on top of the tines 145 to retain them against the lead body 15. For some materials using a solvent or liquid may cause the band 17 to expand or improve its elongation capabilities which may make it easier to place over the tines. When the band dries out it may shrink and constrict further upon the tines. Such an embodiment may also advantageously not result in any additional diameter increase to the over-all lead body diameter.

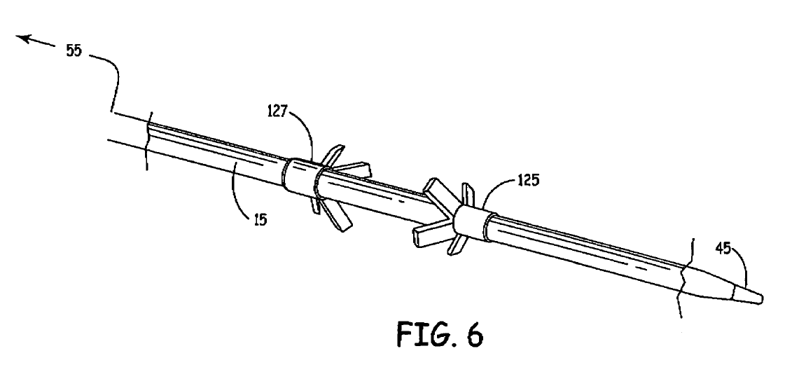

In one embodiment of the invention, one or more tine elements can be forward facing. FIG. 6 illustrates an example of such an embodiment. In this embodiment, the tine element 125 has an angle that is directed backwards, towards the proximal end 55 of the lead body. Such a tine is referred to herein as a backward facing tine. Conversely, the tine element 127 in this embodiment has an angle that is directed forwards, or towards the distal end 45 of the lead body. Such a tine is referred to as a forward facing tine. Embodiments of the invention can have all backward facing tines, all forward facing tines, or some combination thereof. Forward facing tines can be utilized in leads of the invention because the tines are temporarily fixed to the lead body for introduction with the temporary fixative. Leads of the prior art, such as those of commonly assigned U.S. Pat. Ser. No. 10/004,723, were required to be backward facing because an introducer was used to implant them. Forward facing tines would impede the advancement of the lead within the introducer. One embodiment of the invention includes at least one forward facing tine and at least one backward facing tine.

As the temporary fixative dissolves, the folded tines attempt to resume their unrestrained angle as shown in FIGS. 1-6 away from the lead body. The relatively light pressure of the tines on the tissue is readily responded to by the body tissue, especially when those pressures are sustained. This may result in the tines eventually fully deploying even if they are initially constrained by the surrounding tissue to provide an even more robust anchoring mechanism.

An embodiment that includes one or more forward facing tines may assist in preventing or diminishing forward (advancing the lead further in past where it was originally placed) lead migration. An embodiment that includes both forward and backward facing tines (such as that depicted in FIG. 6) may assist both in preventing backward and forward migration of the lead, thereby more securely maintaining it more precisely in an optimal position. It is thought that the backward facing tines prevent backward migration because backwards motion on the lead results in the tines trying to extend further out and grabbing tissue. This causes the tine(s) to maximally resist relative motion. Forward motion of the lead, on the other hand, may result in a collapsing of the backward facing tines towards the lead body which may allow the lead to more easily move forward. It may also be possible, in leads with only backward facing tines, if the tissue surrounding the tines is repeatedly compressed and relaxed to produce a pumping effect on the tine—i.e. driving the lead even further into the tissue.

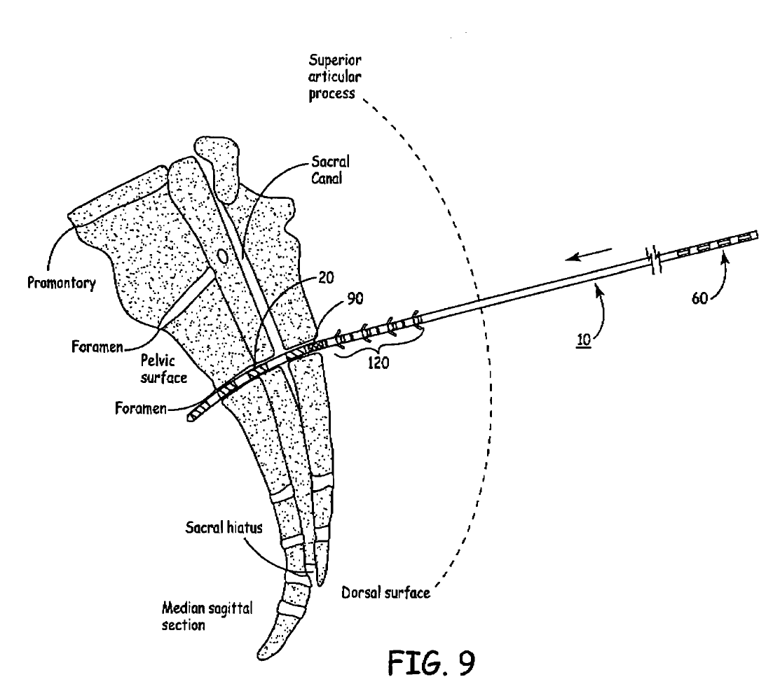

It is within the scope of the present invention to form the tine elements 125, 130, 135 and 140 or 125′, 130′, 135′ and 140′ and 225, 230, 235, and 240 as a single structure with a common tine mounting band extending the length of the tine element array 120 or as an integral section of the outer sheath of the lead body 15 extending through the length of the tine element array 120. Furthermore, whereas the depicted number of tines are equal in number, it will is also within the scope of the invention to make the number of tines not equal among the tine elements. For example, one tine element, could have one tine, another tine element could have two tine elements, and a further tine element could have four tine elements for example. Moreover, whereas the number of tine elements are depicted as equally spaced in the tine element array, the spacing can be varied. It may be desirable to include one or more tine element more proximally disposed along the lead body to be disposed proximally to the bend depicted in the lead body in FIG. 9 to aid in securing the lead or preventing dislodgement of the stimulation electrodes.

One of skill in the art having read this specification will understand that variations of the electrodes and tines are contemplated and encompassed by this invention. For example, different types or lengths of electrodes could be utilized, radially offset tines could be used, and any combination of forward and backward facing tines could be utilized. Further variations of the disclosed embodiments will occur to those of skill in the art.

One embodiment of the invention includes a method of implantation that includes percutaneously inserting a lead of the invention having one or more tines, with the one or more tines folded inward and fixed inward with temporary fixative through the dorsum and the sacral foramen (a singular foramina) of the sacral segment S3 for purposes of selectively stimulating the S3 sacral nerve. The lead can be advanced through the lumen of a hollow spinal needle extended through the foramen, the distal tip of the electrode is positioned adjacent the selected sacral nerve. Stimulation energy can be applied through the lead to the electrode to test the nerve response. The electrode can be moved back and forth to locate the most efficacious location. In one embodiment, a stiffening stylet 100 can be inserted through the lead body lumen to stiffen the lead 10 as it is advanced into and through the tissue. In one embodiment, visual and/or radiographic imaging bands 90 and 95 can be formed around the lead body 15 distal to and proximal to, respectively, the tine element array 120 to be employed in determining the location of the tine element array 120 within the tissue.

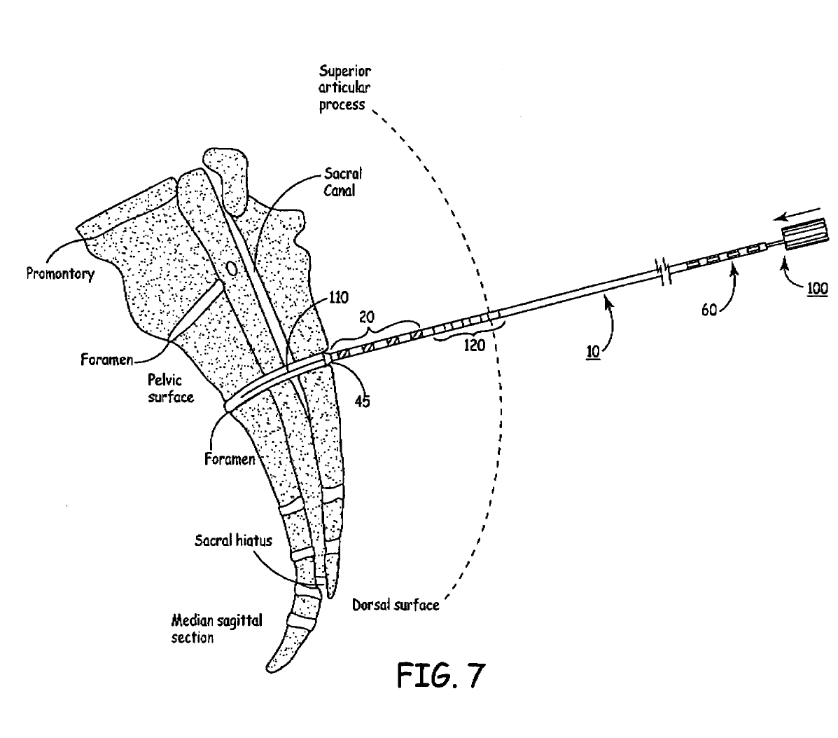

One embodiment of a method of the invention is depicted in FIGS. 7-10, which show exemplary steps of implanting a sacral nerve stimulation lead 10 of the invention and variations thereof described herein. The stylet 100 can be disposed within the lead body lumen so that its distal tip closes the lumen distal end opening. The assembly is advanced percutaneously at a selected angle until the introducer distal end of the guide wire 110 is disposed at the selected foramen as shown in FIG. 7.

To determine the best location of the stimulation electrodes, an insulated needle with both ends exposed for electrical stimulation can be used to locate the foramen and locate the sacral nerve by applying electrical stimulation through the needle using an external pulse generator. The efficacy of the location can be tested by evaluating the physiologic response in relation to the electrical threshold energy required to elicit the response. For control of incontinence, the physician can implant the medical electrical lead 10 near the S3 sacral nerves. The implantable medical electrical lead 10 may, however, be inserted near any of the sacral nerves including the S1, S2, S3, or S4, sacral nerves accessed via the corresponding foramen depending on the necessary or desired physiologic response.

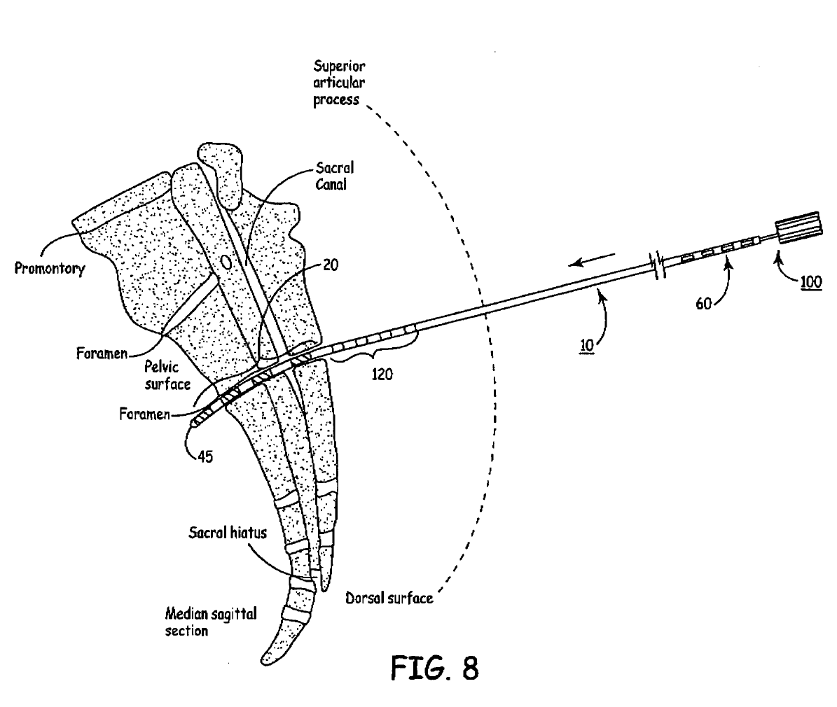

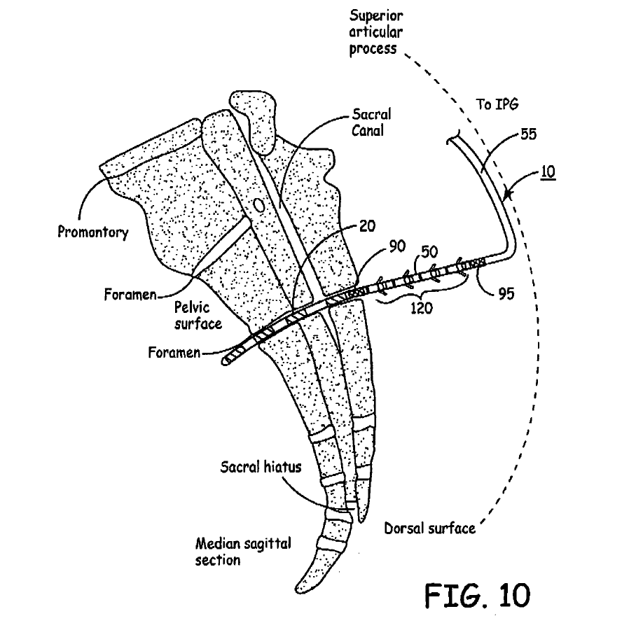

The lead 10, optionally stiffened by the stiffening stylet 100 disposed in the lead lumen, is advanced so that the stimulation electrode array 20 and the tine element array 120 are disposed in relation to the sacral nerve accessed through the foramen and in the subcutaneous tissue, respectively. This is exemplified by FIG. 8 where the lead is placed through the foramen from the posterior entrance into casual contact with the more anterior sacral nerve. After electrical testing to establish optimal positioning is completed, the tine elements are allowed to remain in the tissue until the temporary fixative is dissolved. FIG. 9 shows exemplifies the lead 10 after the lead stylet 100 has been removed, the temporary fixative has been dissolved and the tine elements of the tine element array 120 have been released. The markers, 90 and 95 may be visualized under fluoroscopy. This allows the physician to know where the lead is placed after the tines have been released from the temporary fixative because marker 90 is distal the tines and marker 95 is proximal the tines. When the tines of each tine element are released in subcutaneous tissue, they bear against the tissue and inhibit retraction of the lead body through the subcutaneous tissue. Forward facing tines inhibit distal migration and backward facing tines inhibit proximal migration. As shown in FIG. 10, the proximal portion 55 of the lead body is bent laterally with respect to the distal portion 50 of the lead body 15 and implanted through a subcutaneously tunneled path to the neurostimulator IPG.

Accordingly, the present invention advantageously provides a unique implantable medical electrical stimulation lead that provides adequate stimulation of the sacral nerves for control of incontinence and other pelvic floor disorders with the sacral nerves, with less sensitivity to placement, and enhanced anchoring techniques. The unique leads simplify the implantation procedure and reduce or eliminate the need to reprogram the stimulation energy level provided by the neurostimulator IPG or to re-position the stimulation electrodes.

The medical electrical leads and procedures of the present invention can be used to stimulate multiple nerves or multiple sides of a single nerve bundle. It should also be understood that although sacral nerve stimulation was exemplified herein, the leads of the invention can be used for other types of nerve stimulation. In addition, the medical electrical lead 10 can also be used as an intramuscular lead where the tines can engage against muscle and assist in preventing dislodgement of the distal electrode(s). This may be useful in muscle stimulation such as dynamic graciloplasty or stomach stimulation for gastroparesis or obesity.

Although the invention has been described in detail with particular reference to a certain embodiments thereof, it will be understood variations and modifications can be effected within the scope of the following claims. Such modifications may include substituting elements or components which perform substantially the same function in substantially the same way to achieve substantially the same result for those described herein.

We claim:

1. An implantable medical electrical lead for electrical stimulation of body tissue comprising:
a lead body extending between lead proximal and distal ends;
at least one tine element comprising at least one flexible, pliant, tine, that is adapted to be folded inward and temporarily secured against the lead body using a temporary fixative; and
at least one electrode, wherein said at least one electrode is distal of the at least one tine element on the lead body.

2. The implantable medical lead according to claim 1, wherein the tines of the tine elements are formed of a flexible bio-compatible plastic or a flexible bio-compatible superelastic alloy.

3. The implantable medical lead according to claim 1, wherein the tines of the tine elements are formed of polyurethane compound, or silicone rubber compound.

4. The implantable medical lead according to claim 1, wherein the temporary fixative comprises sugar, sugar alcohol, sugar cellulose, protein, acrylic resin, alkyd resin, or a material made from animal intestines.

5. The implantable medical lead according to claim 4, wherein the temporary fixative comprises glucose, mannitol, polysaccharide glucose, albumin, polyglycolic acid, polyglactin, polydioxone, or polyglyconate.

6. The implantable medical lead according to claim 1, wherein the temporary fixative covers only a portion of the distal end of the lead body.

7. The implantable medical lead according to claim 1, wherein the temporary fixative creates a band that is positioned over the tines of at least one of the tine elements.

8. The implantable medical lead according to claim 7, wherein the temporary fixative comprises animal intestines, polyglycolic acid, polyglactin, polydioxone, or polyglyconate.

9. The implantable medical lead according to claim 7, wherein each tine element has an individual band positioned over the tine element.

10. The implantable medical lead according to claim 7, wherein there is one band positioned over the one or more tine elements.

11. The implantable medical lead according to claim 1, wherein the tines of at least one of the tine elements are angled forward.

12. The implantable medical lead according to claim 1, wherein there are at least four tine elements.

13. The implantable medical lead according to claim 1, wherein there is at least one tine element with forward facing tines and at least one tine element with backward facing tines.

14. A medical electrical stimulation system comprising:
an implantable pulse generator for providing medical electrical stimulation; and
a medical electrical lead coupled to the implantable pulse generator for electrical stimulation of body tissue, the medical electrical lead comprising
a lead body extending between lead proximal and distal ends;
at least one tine element comprising at least one flexible, pliant, tine, that is adapted to be folded inward and temporarily secured against the lead body using a temporary fixative; and
at least one electrode, wherein said at least one electrode is distal of the at least one tine element on the lead body.

15. The system according to claim 14, wherein the tines of the tine elements are formed of a flexible bio-compatible plastic or a flexible bio-compatible superelastic alloy.

16. The system according to claim 14, wherein the tines of the tine elements are formed of polyurethane compound, or silicone rubber compound.

17. The system according to claim 14, wherein the temporary fixative comprises sugar, sugar alcohol, sugar cellulose, protein, acrylic resin, alkyd resin, or a material made from animal intestines.

18. The system according to claim 17, wherein the temporary fixative comprises glucose, mannitol, polysaccharide glucose, albumin, polyglycolic acid, polyglactin, polydioxone, or polyglyconate.

19. The system according to claim 14, wherein the temporary fixative covers only a portion of the distal end of the lead body.

20. The system according to claim 14, wherein the temporary fixative creates a band that is positioned over the tines of at least one of the tine elements.

21. The system according to claim 20, wherein the temporary fixative comprises animal intestines, polyglycolic acid, polyglactin, polydioxone, or polyglyconate.

22. The system according to claim 20, wherein each tine element has an individual band positioned over the tine element.

23. A method of providing electrical stimulation of body tissue at a stimulation site employing an implantable pulse generator comprising:
providing an implantable medical lead comprising:
a lead body extending between lead proximal and distal ends;
at least one tine element comprising at least one flexible, pliant, tine, that is adapted to be folded inward and temporarily secured against the lead body using a temporary fixative;
at least one electrode, wherein said at least one electrode is distal of the at least one tine element on the lead body;
at least one proximal connector element formed in a connector array in a proximal segment of the lead body;
percutaneously introducing the implantable medical lead adjacent to the stimulation site;
allowing the temporary fixative to dissolve, thereby allowing the at least one tine element to fold outward; and
coupling the at least one proximal connector element with the implantable pulse generator.

24. The method according to claim 23 further comprising the step of using an insulated needle with both ends exposed to apply electrical stimulation through the needle using an external pulse generator in order to determine the best location for the at least one electrode.

25. The method according to claim 23 further comprising the step of testing the efficacy of the location.

26. The method according to claim 25, wherein the step of testing the efficacy of the location is accomplished by evaluating the physiologic response in relation to the electrical threshold energy required to elicit the response.
